(19) United States
(12) Patent Application Publication
(10) Pub. No.: US 2009/0118352 A1
(43) Pub. Date: May 7, 2009

(54) Broad spectrum microbicidal and spermicidal compositions and methods

(75) Inventors: Kewang Lu, Dover, DE (US); Qiong Gong Ning, Kunming (CN); Lei Zhang, Kunming (CN)

Correspondence Address: KEWANG LU, 16 CHADWICK DRIVE, DOVER, DE, 19901, US

(73) Assignee: ESAWTECH, LTD., DOVER, DE (US)

(21) Appl. No.: 12/290,288

(22) Filed: Oct. 29, 2008

Related U.S. Application Data
(60) Provisional application No. 61/001,936, filed on Nov. 7, 2007.

(51) Int. Cl. A61K 31/4015; A61P 31/12; A61P 31/04

(52) U.S. Cl. 514/423

(57) ABSTRACT

The present invention relates to microbicidal and spermicidal compositions, methods and topical pharmaceutical compositions containing N-cocoyl amino acid pyrrolidone carboxylates (N-cocoyl amino acid pyrrolidone salts, CAPS) as active ingredients. The invention is for the prevention and control of sexually transmitted diseases and for the prevention of pregnancy. The microbicidal spectrum of the invention includes various STD pathogens, HIV viruses as well as gram positive and negative bacteria, and yeasts. Spermicidal barriers containing the compositions are also provided.

The N-cocoyl amino acid pyrrolidone salts is represented by the formula:

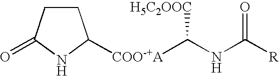

Wherein A is an amino acid residue and R is a C.sub.1O-C.sub.14 fatty acid residue

### RELATED APPLICATION

This application is based on, and claims benefit of, U.S. Provisional Application Ser. No. 61/001,936, filed on Nov. 7, 2007.

### FIELD OF THE INVENTION

The present invention relate to the topical application of a novel composition containing N-cocoyl amino acid pyrrolidone salts for preventing or reducing the transmission of sexually transmitted diseases (STDs) in sexually active individuals. This invention also relates to compositions and methods for preventing conception and/or reducing the risk of conception in sexually active females. The present compositions and methods are especially effective for preventing of sexually transmitted diseases during male/female sexual intercourse. It can, however, be used by heterosexual, homosexual, and bisexual individuals to reduce the risk of being infected by, or of transmitting, a sexually transmitted disease through sexual contact.

The method of this invention comprises application of an effective amount of the microbicidal formulation within the vagina or anorectic region with choice of different dosage form applications. The invention also relates to methods of inhibiting the activity of other pathogens whose mode of transmission is nonsexual. The compositions of use in the invention are also effective in the inhibition of bacteria and fungi, which coexist with viruses or viral infections.

### BACKGROUND OF THE INVENTION

Sexually transmitted diseases (STDs) are among the most prevalent and communicable diseases, and continue to be a significant public health problem. It is estimated that more than 250 million people worldwide, and close to 3 million people in the United States, are infected annually by gonorrhea. Annual worldwide incidence of syphilis is estimated at 50 million people, with 400,000 in the United States annually needing treatment. The human immunodeficiency virus (HIV), resulting in fatal acquired immunodeficiency syndrome (AIDS), has spread rapidly in both homosexual and heterosexual groups. For many sexually transmitted infections, vaccines do not exist, and therapeutic agents are only partially effective, expensive, and difficult to distribute. In addition, female partners in many relationships do not control pregnancy or STI. One approach to the general control of STDs is the use of topically applied, female controlled microbicides that inactivate the relevant pathogens.

It is thought that certain bacteria known to cause STD's may aid in HIV transmission. In persons who have been exposed to HIV, certain bacteria that cause STD's often fail to respond to therapies that are otherwise highly effective. HIV infection may help the spread of a bacterial STD that in turn helps to spread HIV. STD's pathogens such as Chlamydeous, syphilis, genital herpes and gonorrhea that cause ulcerations of the genital skin seem to increase the risk of acquiring or transmitting HIV infection sexually.

### DESCRIPTION OF THE PRIOR ART

NONOXYL-9 (N-9) is present in a large number of condoms and other spermicidal agents. Recent research, however, has found that N-9 can disrupt epithelial cells in the vagina, thereby increasing the risk of HIV infection. Frequent use of N-9 is also positively correlated with bacterial vaginosis, genital ulcers and Vulvitis, vaginal Candidiasis, toxic shock syndrome, and epithelial disruption of the cervix and the vagina. The final phase III multi-centre randomised placebo-controlled trial of N-9, undertaken by the United Nations Joint Programme on HIV/AIDS in 2002, showed that N-9 had no efficacy in preventing HIV transmission. Indeed, the transmission rate was marginally higher in the N-9 group. In 2003 the World Health Organization (WHO) recommended that people not use N-9 for protection against HIV/AIDS or other STIs. (Van Damme L, et al. (2002) Effectiveness of COL-1492, a nonoxynol-9 vaginal gel, on HIV-1 transmission in female sex workers: A randomised controlled trial. Lancet 360:971-977.; Stephenson J (2000) Widely used spermicide may increase, not decrease, risk of HIV transmission. Jama 284(8): 949.)

U.S. Pat. No. 5,004,757 is directed to a method of deactivating viruses on surfaces by applying a three-part composition containing glutaraldehyde. The composition also contains hydrogen-bonded glycol molecules to eliminate aldehyde odor, and an anionic surfactant such as sodium dodecyl sulfate (SDS) as a potentiator of the virucidal activity of the glutaraldehyde component. The patent indicates that SDS has limited virucidal activity on its own, but has a synergistic effect when combined with gluteraldehyde. Due to the fact that gluteraldehyde is a well-known mutagen, the invention is not useful with direct contact of human epithelium tissues.

U.S. Pat. Nos. 6,063,773, 7,078,392 presented a method for treating and preventing various infections, including papilloma virus and fungal and parasitic infections. The inventions relate to use sulfated polysaccharide, such as cellulose sulfate (a high molecular weight carboxymethyl cellulose-based polymer), for treating and preventing various sexual infections. The cellulose sulfate formulations were tried clinically for preventing HIV transmissions. In 2007, World Health Organization and UNAIDS has prematurely stopped the clinical trial of using cellulose sulfate to prevent HIV transmission in women because of a higher number of HIV infections in the active group compared with the placebo group. (Lut Van Damme et al. Lack of Effectiveness of Cellulose Sulfate Gel for the Prevention of Vaginal HIV Transmission, N Engl J med 359:463-472; Statement by the world Health Organization and UNAIDS: Cellulose sulfate microbicide trial stopped, 31 Jan. 2007)

U.S. Pat. Nos. 5,314,917 and 6,297,278 provided an antimicrobial composition for inhibiting the activity of enveloped viruses, treatment of viral infections and method for inactiviting sperm. The composition comprises: a mixtures of a betaine, an amino oxide and a protonating agent to adjust the pH of the overall composition to about 5.5 or below (C31G). Clinical trials showed that C31G was associated with higher reporting of reproductive adverse events. The data were insufficient to conclude whether C31G is effective at preventing HIV infection relative to placebo. (Feldblum P J, et al. (2008) SAVVY Vaginal Gel (C31G) for Prevention of HIV Infection: A Randomized Controlled Trial in Nigeria. PLoS ONE 3(1): e1474. Peterson L, et al. (2007) SAVVY(R) (C31G) Gel for Prevention of HIV infection in Women: A Phase 3, Double-Blind, Randomized, Placebo-Controlled Trial in Ghana. PLoS ONE 2(12): e1312.)

U.S. Pat. No. 5,617,877 introduced an acidic buffer gel contained within a flexible dome device positioned over the uterine cervix for controlling pH in the vagina for in-activating both sperm and STIs. The device is made of a dome and the absorbent acidic buffer gel is contained within the dome. The buffer gel (BufferGel™) is reported to be a negatively charged, non-absorbable high molecular weight polymer gel, designed to maintain vaginal pH below 5 in the presence of semen. BufferGel relies on a polymer comprised of carboxylated monomers to control the vaginal pH. BufferGel is placed over the uterine cervix that allowing the STD-causing microbes to readily migrate throughout the lower genital tract. The use of devices in combination with BufferGel requires significant skill and motivation by the user to obtain, and maintain, proper placement of the device. It is important to mention that some of the pathogens will survive the low pH environment. The efficacy of the acidic gel is solely depends on the acidity of the gel, and it might not guarantee a desirable effect on all the STI pathogens.

U.S. Pat. Nos. 7,226,914 and 7,235,536 relates to the treating, inhibiting or preventing of certain infectious agents including papilloma virus and various vaginitis-causing microbes by employing cellulose sulfate and other sulfated polysaccharides. Carraguard is used as a microbicidal gel containing a sulfated polysaccharide. Resent clinical trial with this sulfated polysaccharides composition as microbicide did not demonstrate that the compound were effective in preventing male-to-female HIV transmission during vaginal intercourse (Microbicide Trials Network: Statement on Carraguard phase III clinical trial findings, Feb. 18, 2008).

U.S. Pat. No. 6,417,144 relate to a solution for cleaning contact lenses. The solution comprises an amino acid derivative and at least one nonionic surfactant. The amino acid derivative is used as a component to increase the cleaning power together with a nonionic surfactant. The solution for contact lenses has an excellent cleaning effect and an adequate safety for the eye. This invention did not mention of preventing HIV and STDs.

### DETAILED DESCRIPTION OF THE INVENTION

The present invention relates to a novel composition and method for the prevention and control of sexually transmitted diseases and for the prevention of pregnancy. The invention also relates to treatment of viral infections. More particularly, the invention relates to a method for inhibiting the development of diseases and infections caused by viruses and some sexually transmitted pathogens whose major mode of transmission is sexual. In other aspects the invention also relates to methods of inhibiting the activity of enveloped viruses and other pathogens whose mode of transmission is nonsexual. The compositions of use in the invention are also effective in the inhibition of bacteria and fungi, which coexist with viruses or viral infections. Additionally, the invention relates to a prevention or treatment for virus related diseases, particularly sexually transmitted diseases related to AIDS, and to diseases related to this and other opportunistic infections of the immune-compromised host.

Transmission of HIV is often associated with the co-transmission of other viral and/or microbial pathogens. HIV may not be the sole agent responsible for AIDS (Duesberg, P. H. (1991) Proc. Natl. Acad. Sci. 88:1575-1579; Lemaitre, M., Guetard, D., Henin, Y., Montagnier, L. and Zerial, A. (1990). Res. Virol. 141:5-16). For this reason, antimicrobial agents, such as those described in this invention, with a broad spectrum of activities against viruses, bacteria, and yeasts may be of particular value in the prevention and treatment of Acquired Immune Deficiency Syndrome (AIDS). It is thought that certain bacteria known to cause STD's may aid in HIV transmission. In persons who have been exposed to HIV, certain bacteria that cause STD's often fail to respond to therapies that are otherwise highly effective. HIV infection may help the spread of a bacterial STD that in turn helps to spread HIV. STD's pathogens that cause ulcerations of the genital skin seem to increase the risk of acquiring or transmitting HIV infection sexually. The compositions described in the invention may also be of use to inactivate other viruses, including vaccinia, varicella, herpes zoster, cytomegalovirus, influenza, mumps and measles.

### APPLICATIONS OF THE INVENTION

The present invention provides a novel composition and method for the prevention and control of sexually transmitted diseases and for the prevention of pregnancy. The invention also relates to treatment of viral infections. More particularly, the invention relates to a method for inhibiting the development of diseases and infections caused by viruses and some sexually transmitted pathogens whose major mode of transmission is sexual. In other aspects the invention also relates to methods of inhibiting the activity of pathogens whose mode of transmission is nonsexual. The compositions of the invention are also effective in the inhibition of bacteria and fungi, which coexist with viruses or viral infections.

The compositions can be formulated in the form of a gel, liquid, aerosol, mist, sponge, spray, foam, gel, cream, salve, jelly, suppository and film. The compositions can be applied to the external genital organs, vagina, anorectic region and rectum in different dosage forms with appropriate apparatus, for example the gel could be applied to vagina or rectum through an applicator or syringe. The compositions of the invention can also be incorporated into douches. The compositions can also be incorporated into wipes.

The invention also provides a method for preventing and the treatment of certain skin diseases. Such as ringworm and other fungi infectious skin diseases.

The invention also provides a broad spectrum and highly efficient microbicidal composition used for reducing the risk of transmission of STD-causing organisms to health care providers and laboratory personnel (or other persons) who may come in contact with biological samples and specimens

The compositions of the present invention can also be used as spermicides. These compositions can be used alone, with other known spermicides and with or incorporated into contraceptive devices such as condoms, sponges, vaginal inserts, contraceptive films, diaphragms, suppositories, contraceptive patches or sustained release devices. For use as spermicides, these compositions of the invention can be applied alone; with other microbicides; and with or incorporated into the contraceptive devices described above.

The composition can also be used in animals as disinfecting or antiseptic agent.

### Synthesis and Preparation of the N-cocoyl Amino Acid Pyrrolidone Salt

The microbicidal composition of the present invention consisting of an amino acid derivative that is modified with containing a C.sub.10-14 alkyl group at the N-terminus, particularly preferably N-cocoyl group is preferred. The amino acid residue bears a positive charge and the negative counter ion is organic molecule, preferably pyrrolidone salts. There are no halogen elements, such as chloride or bromide, in the structure of the amino acid compound. One general example is an amino acid ethyl ester acylated at the N-terminus with a coconut oil fatty acid residue, and having DL-pyrrolidone carboxylic acid added thereto.

The N-cocoyl amino acid pyrrolidone salts represented by the formula:

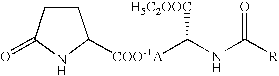

Wherein A is an amino acid residue and R is a C.sub.10-C.sub.14 fatty acid residue. In the structure, the amino acid residue could be from that of an arginine, lysine, and histidine; homolysine, or an unnatural amino acid residue bearing a positive charge, and other di or tri-peptide bearing a positive charge group.

For example, the N-cocoyl Arginine pyrrolidone salts is prepared with the following procedures:

Preparation solution A: Mix 1.2 equivalent N,N′-Dicyclohexylcarbodiimide (DCC) with 1 equivalent myristic acid (CH₃(CH₂)₁₀COOH) in DMF and stir for 45 minute to 2 hours, Add in 1.2 equivalent N-Hydroxylsuccinimide (NHS) dissolved in CH₂Cl₂, keep stirring until precipitate forms, which is the undesired side product dicyclohexane urea. Filter the reaction mixture to remove the precipitate urea, and save the solution as solution A.

Preparation solution B: L-Arginine ethyl ester dihydrochloride and 1 equivalent of NaOH at 0 degree C. in DMF or CH₂Cl₂ for 30 minutes, and then add 1.3 equivalent of triethyl amine (Et₃N). Mix to a homogeneous solution and save as solution B.

Preparation of the amino salt: Mix solution A and B together at room temperature for 2 hours. Remove most of the solvent by vacuum. Dissolve the product in methanol and add one equivalent of NaOH, and extract the product with a mixture of water and ether/EtOAc. Save and dry the organic phase under vacuum. Re-precipitate the product by using solvent hexane or water or methanol, or a mixture of methanol and water. Re-dissolve the product in methanol, and add one equivalent of DL-pyrrolidone carboxylic acid, and then remove the methanol under vacuum to obtain the final product.

In the formulation of the present invention the N-cocoyl amino acid pyrrolidone salts is contained usually in a proportion of from 0.003 to 3.0% (w/w).

The N-cocoyl amino acid pyrrolidone salts are soluble in water and highly safe with respect to acute toxicity (LD.sub.50). It has no irritation to skin and ophthalmic mucosa. Moreover, it has good biodegradable characteristic and can rapidly be decomposed in wastewaters.

### Preparation of the Compositions

In the microbicide compositions of the present invention a natural plant polysaccharides could be incorporated in the formula to enhance the antibacterial effect and protect the epithelium tissues. The plant polysaccharides may, for example, be Aloe Vera polysaccharides.

In the microbicide compositions of the present invention an ampholytic surfactant may be added and incorporated so long as it does not inhibit the effect obtained by the N-cocoyl amino acid pyrrolidone salts, in an amount which does not impair the antimicrobial effect. The ampholytic surfactant may, for example, be an amine oxide such as alkyl dimethyl amine oxide.

In the present invention, in addition to the specific active ingredient, one or more additive components conventionally used may further be incorporated in a conventional amount, as the case requires. The additive can be used including natural polysaccharides, natural plant acid and their salts, fragrance, colorant, flavor, plasticizer, stabilizing agent, emulsifier or moisturizer. Such an additive component is preferably one, which has a high safety for the human body, which is adequately compatible with active ingredients, and which has no influence over the effectiveness and stability of the composition.

In order to facilitate a further understanding of the invention, the following examples are presented primarily for the purposes of illustrating more specific details thereof. The invention is not to be deemed as limited thereby except as defined in the claims.

### EXAMPLE 1

The liquid microbicide composition of the present invention is prepared by adding and incorporating the active ingredients in proper amounts into a proper aqueous medium in an optional order in a conventional method:

Mix the following ingredients (w/w) to obtain a homogeneous solution

| | |
|---|---|
| N-cocoyl amino acid pyrrolidone salts: | 0.003-3.0, |
| Water: | 10.0-99.997. |

When plant polysaccharides (powder of freeze dried Aloe Vera gel)
is added in the composition, add the following ingredients (w/w)
and mix to obtain a homogeneous solution

| | |
|---|---|
| Plant polysaccharides: | 0.001-4.0 |
| N-cocoyl amino acid pyrrolidone salts: | 0.003-3.0 |
| Water: | 10.0-99.995 |

The gel microbicide composition of the present invention is prepared by the following procedures:
A. Mix the following ingredients (w/w) and heat to 60° C.-95° C. with stirring to obtain a homogeneous solution

| | |
|---|---|
| Ampholytic surfactant: | 0.0016-3.0 |
| Aloe Vera: | 0.001-4.0 |
| Water | 10.0-99.999 |

B. Add Hydroxypropyl ethyl cellulose (w/w) 0.010-5.0 to the 60° C.-95° C. homogeneous solution, stir for more than 30 minutes, stops heating, slowly reduces temperature to 50° C.
C. Add the N-cocoyl amino acid pyrrolidone salts 0.003-3.0 to the above solution,

Keep stirring until the mixture's temperature dropped to room temperature to obtain a colorless transparent gel.

An appropriated amount (3-5 ml) of gel was filled into a syringe. The filled syringe was sealed within an aluminum foil bag. At use, the user open the foil bag and insert the syringe to vagina or rectum to apply the material.

Table 1 listed 37 formulations. They are prepared following the above procedures to make liquid or gel preparations.

TABLE 1
FORMULATIONS OF MICROBICIDES (W/W)

| Formulation | AV | C1 | H₂O | CAPS | HP |
|---|---|---|---|---|---|
| 1 | 1.00 | 1.00 | 94.50 | 1.50 | 2.00 |
| 2 | 0.00 | 0.00 | 99.40 | 0.60 | 0.00 |
| 3 | 0.25 | 0.00 | 99.08 | 0.67 | 0.00 |
| 4 | 0.0013 | 0.002 | 99.995 | 0.0034 | 0.01 |
| 5 | 0.25 | 0.33 | 96.75 | 0.67 | 2.00 |
| 6 | 0.006 | 0.008 | 99.92 | 0.017 | 0.05 |
| 7 | 0.013 | 0.017 | 99.84 | 0.034 | 0.10 |
| 8 | 0.003 | 0.003 | 99.9 | 0.007 | 0.02 |
| 9 | 0.005 | 0.007 | 99.9 | 0.013 | 0.04 |
| 10 | 0.025 | 0.033 | 99.7 | 0.067 | 0.200 |
| 11 | 0.003 | 0.004 | 99.96 | 0.008 | 0.025 |
| 12 | 1.50 | 0.50 | 50.00 | 1.00 | 0.50 |
| 13 | 2.00 | 0.0016 | 90.0 | 3.00 | 5.0 |
| 14 | 0.20 | 0.25 | 95.50 | 0.50 | 0.30 |
| 15 | 0.00 | 0.00 | 10.00 | 0.003 | 0.00 |
| 16 | 0.00 | 0.00 | 99.995 | 0.003 | 0.00 |
| 17 | 0.00 | 0.00 | 98.50 | 1.5 | 0.00 |
| 18 | 0.00 | 0.00 | 97.00 | 3.0 | 0.00 |
| 19 | 4.0 | 0.00 | 96.00 | 0.003 | 0.00 |
| 20 | 1.8 | 0.00 | 97.00 | 1.2 | 0.00 |
| 21 | 0.001 | 0.00 | 97.00 | 3.0 | 0.00 |
| 22 | 0.00 | 3.0 | 97.00 | 0.003 | 0.00 |
| 23 | 0.00 | 1.7 | 97.00 | 1.3 | 0.00 |
| 24 | 0.00 | 0.0016 | 97.00 | 3.0 | 0.00 |
| 25 | 0.00 | 0.00 | 95.00 | 0.003 | 5.0 |
| 26 | 0.00 | 0.00 | 96.30 | 1.4 | 2.3 |
| 27 | 0.00 | 0.00 | 96.99 | 3.0 | 0.01 |
| 28 | 0.001 | 0.00 | 97.00 | 0.003 | 0.00 |
| 29 | 2.0 | 1.5 | 94.90 | 1.6 | 0.00 |
| 30 | 4.0 | 0.0016 | 93.00 | 3.0 | 0.00 |
| 31 | 0.00 | 3.0 | 96.99 | 0.0033 | 0.01 |
| 32 | 0.00 | 1.3 | 94.50 | 1.7 | 2.5 |
| 33 | 0.00 | 0.00 | 92.00 | 3.0 | 5.0 |
| 34 | 0.001 | 0.00 | 99.995 | 0.0033 | 0.01 |
| 35 | 2.2 | 0.00 | 93.20 | 1.8 | 2.8 |
| 36 | 4.0 | 0.00 | 88.00 | 3.0 | 5.0 |
| 37 | 0.0 | 0.0 | 99.5 | 0.5 | 0.0 |

Note:
AV = Freeze dried Aloe Vera gel powder;
C1 = Alkyldimethylamine oxide;
H₂O = Water;
CAPS = N-cocoyl Arginine pyrrolidone salts;
HP = Hydroxypropyl methyl cellulose.

### EXAMPLE 2

MICROBICIDE COMPOSITION OF SUPPOSITORIES

| | |
|---|---|
| CAPS | 4.8 g |
| Alkyldimethylamine oxide | 2.4 g |
| Aloe Vera powder | 1.8 g |
| Sodium phytate | 1.8 g |
| Glycerogelatin | 900.0 g |
| Glycerin | 20.0 g |
| Water | 80.0 g |

Dissolve N-cocoyl Arginine pyrrolidone salts (CAPS), sodium phytate and freeze dried Aloe Vera powder in water, add glycerin and mix to a homogeneous solution, Add the above to Glycerogelatin, mix at 45° C. to homogeneous, and injected to molds, cooled and ejected for packing. Each one weights 4 gm.

### EXAMPLE 3

MICROBICIDE COMPOSITIONS OF MIST AND SPRAY

| Ingredients | Spray | Mist |
|---|---|---|
| CAPS | 0.6 kg | 0.8 g |
| Alkyldimethyl amine oxide | 0.0 kg | 0.4 g |
| Isopropylene | 5.0 kg | 5.0 g |
| Dimethyl Ether | 0.0 kg | 20.0 g |
| Water | 94.4 kg | 80.0 g |

Spray: Dissolve N-cocoyl Arginine pyrrolidone salts (CAPS) in water, slowly add isopropylene and mix to homogeneous solution.; package in container with spray device.
Mist: Dissolve N-cocoyl Arginine pyrrolidone salts (CAPS) and Alkyldimethyl amine oxide in water, slowly add isopropylene and mix to homogeneous solution; add Dimethyl Ether and fill in container with spray device.

### EXAMPLE 4

MICROBICIDE COMPOSITION OF FILM

| | |
|---|---|
| CAPS | 0.80 kg |
| Aloe Vera | 0.16 kg |
| Gelatin | 6.20 kg |
| Hydroxyethyl cellulose | 0.50 kg |
| Glycerin | 31.00 kg |
| Water | 25.50 kg |

Triturate Gelatin with Hydroxyethyl cellulose and glycerin. Mix thoroughly to form a slurry. Dissolve N-cocoyl Arginine pyrrolidone salts (CAPS) in warm water to obtain a homogeneous solution. Add the solution to the above slurry. Heat the mixture at 40° C. and mix until cellulose and gelatin are completely hydrated. Pour the solution on polyethylene sheet to cast a film of about 2 mm thick to be cut after cooling at size 3.9 × 3.9 cm.

### EXAMPLE 5

### Effects of the Microbicide on Ureaplasma Urealyticum

### 1. Materials

1) Testing strain: Ureaplasma Urealyticum: clinical strain
2) Culture medium: High efficiency Ureaplasma culture medium;
3) Testing sample: Microbicide solution prepared as Example 1, formulation 3.
   2. Testing Methods and Standards: [symbol] Technical Standard for Disinfection [symbol] (2002 Edition) Issued by “The Health Department of China”
1) The tests followed the “Standard” with consideration of the characteristics of Ureaplasma urealyticum
2) Testing Temperature: 20° C.˜25° C.
3) Concentration of sample dilution: 1:100; 1:200; 1:500; 1:1000
4) Concentration of Ureaplasma Urealyticum: 10⁷
5) Treatment: Ureaplasma Urealyticum suspension:diluted sample=1:9
6) Reacted time: 1 min.; 3 min.; 5 min.;
7) Diluting methods were used to remove the residue of the sample
8) Tests repeated for two times
   3. Results: The Killing Effects of sample to Ureaplasma Urealyticum (Table 2)

TABLE 2
The Effects of Testing Sample on Ureaplasma Urealyticum

| time | Dilution of Sample | | | |
|---|---|---|---|---|
| (min.) | 1:100 | 1:200 | 1:500 | Control |
| 1 | − | + | + | + |
| 3 | − | − | + | + |
| 5 | − | − | + | + |

Note:
Control: Ureaplasma Urealyticum growth observed;
+: Ureaplasma Urealyticum growth observed;
−: No Ureaplasma Urealyticum growth observed.

### 4. Conclusion

The testing results showed: Testing Sample diluted for 100 times and reacted with Ureaplasma Urealyticum suspension for 1 minute killed Ureaplasma Urealyticum.

### EXAMPLE 6

### Effects of the Microbicide on Neisseria Gonorrhoeae

### 1. Materials

1) Testing strain: Neisseria Gonorrhoeae, clinical strain
2) Culture medium: 10% blood dish;
3) Testing sample: Microbicide solution prepared as Example 1, formulation 3.
   2. Testing Methods and Standards: [symbol] Technical Standard for Disinfection [symbol] (2002 Edition) Issued by “The Health Department of China”
1) The tests followed the “Standard” with consideration of the characteristics of Neisseria gonorrhoeae
2) Temperature: 25° C.˜28° C.
3) Concentration of sample prepared: 1:25; 1:50; 1:100
4) Neisseria gonorrhoeae suspension: 10⁷
5) Treatment: Neisseria gonorrhoeae suspension:diluted sample solution=1:1
6) Treatment time: 1 min.; 3 min.; 5 min.;

### 3. Results

The Killing Effect of Testing Sample on Neisseria Gonorrhoeae (Table 3)

TABLE 3
Effects of Testing Sample on Neisseria Gonorrhoeae

| React time | Dilution of Sample | | | |
|---|---|---|---|---|
| (min.) | 1:25 | 1:50 | 1:00 | Control |
| 1 | − | − | − | + |
| 3 | − | − | − | + |
| 5 | − | − | − | + |

Note:
+: growth of Neisseria gonorrhoeae observed
−: No growth of Neisseria gonorrhoeae

### 4. Conclusion

The testing results showed: Testing Sample diluted for 100 times and reacted with Neisseria gonorrhoeae suspension for 1 minute, killed Neisseria gonorrhoeae.

### EXAMPLE 7

Effects of the Microbicide on Trichomonas vaginalis

### 1. Materials

1) Testing strain: Clinical isolated Trichomonas vaginalis strain
2) Culture medium: liver infusion medium;
3) Testing sample: Microbicide solution prepared as Example 1, formulation 3.
   2. Testing Methods and Standards: [symbol] Technical Standard for Disinfection [symbol] (2002 Edition) Issued by “The Health Department of China”

### 3. Results

The Killing effect of testing sample to Trichomonas vaginalis (Table 4)

TABLE 4
Effects of Testing Sample on Trichomonas Vaginalis

| time | Dilution of sample | | | Control |
|---|---|---|---|---|
| (minutes) | 1:50 | 1:100 | 1:200 | 1:50 |
| 1 | − | − | + | + |
| 3 | − | − | + | + |
| 5 | − | − | + | + |

Note:
+: Active Trichomonas vaginalis
−: Trichomonas vaginalis were not active and were dissolved

### 4. Conclusion

The testing results showed: Testing Sample diluted for 100 times and reacted with Trichomonas vaginalis suspension for 1 minute killed Trichomonas vaginalis.

### EXAMPLE 8

### Effects of the Microbicide on HIV-1

### 1. Materials

1) Human Immunodeficiency Virus: HIV-1 IIIB strain;
2) Cell: MT 4 cell;
3) Testing sample: Microbicide solution prepared as Example 1, formulation 2.
   2. Testing Methods and Standards: [symbol] Technical Standard for Disinfection [symbol] (2002 Edition)
1) Diluted the Testing sample to 1:20; 1:40; 1:80 and 1:160 solutions with distilled water.
2) Added 0.9 ml sample (1:20; 1:40; 1:80, 1:160) to 0.1 ml HIV suspension solution respectively
3) The mixtures were reacted for 1 min.; 3 min.; 5 min. at room temperature
4) Take 0.1 ml each of the above and dilute with culture fluid to 1:1000
5) The virus concentration were titrated (96 well plate, each well contains 0.1 ml of 50000/ml MT4 cell)
6) Incubated for 7 days (37° C., 5% CO₂)
7) Added 0.1 ml of fresh culture fluid to each well at the third day
8) Observe the cell growth everyday
9) Evaluate the existence of Virus with CPE

### 3. Experimental Groups

1) Testing group
2) Drug (sample) removed control group (1000 times diluted Virus+1000 times diluted sample)
3) Normal cell control group
4) Virus control group (Virus+culture fluid)

### 4. Results

HIV-1 titration was 10⁷ TCID₅₀.

The “drug removed control group” showed that the dilution could stop the effect of sample to the virus.

The Testing sample at 1:20 and 1:40 dilution reacted with HIV-1 for 1 min, titrated and cultured with MT4 cell, no cell abnormality were observed

The Testing sample at 1:80 dilution reacted with HIV-1 for 3 min., titrated and cultured with MT4 cell, no cell abnormality were observed.

The Testing sample at 1:160 dilution reacted with HIV-1 for 5 min., titrated and cultured with MT4 cell, no cell abnormality were observed.

### 5. Conclusion

Testing sample diluted at 1:20 (Example 1, formulation 15) and 1:40 solutions reacted with HIV-1 suspension for 1 min; diluted at 1:80 solution reacted with HIV-1 suspension for 3 min; diluted at 1:160 solution reacted with HIV-1 suspension for 5 min, all the kill rates were 100%.

### EXAMPLE 9

The Minimum Bactericide Concentration (MBC) of Testing Sample to Candida albicans

### 1. Materials

1) Testing strain: Candida albicans ATCC 10231, the 4^{th} generations.
2) Testing sample: Microbicide solution prepared as example 1, formulation 2

### 2. Testing Method

Nutrient broth dilution method, Temperature 21±1° C.

### 3. Testing Results (Table 5)

TABLE 5
The MBC of Testing sample to Candida albicans

| | | | | | | | | | | | | Bacterial |
|---|---|---|---|---|---|---|---|---|---|---|---|---|
| | MBC of Sample dilutions | | | | | | | | | Positive | Negative | concentration |
| test | 1 | 2 | 4 | 8 | 16 | 32 | 64 | 128 | 256 | control | control | (CFU/ml) |
| 1 | − | − | − | − | − | + | + | + | + | + | − − | 2.13 × 10⁶ |
| 2 | − | − | − | − | − | + | + | + | + | | | 1.89 × 10⁶ |

Note:
“+” bacterial growth observed;
“−” no bacterial growth observed.

### 4. Conclusion

To Candida albicans the MBC of testing sample is it's 16 times dilution (2 min).

### EXAMPLE 10

### Effects of the Microbicide on HIV-1

### 1. Materials

1) Human Immunodeficiency Virus: HIV-1 IIIB strain;
2) Cell: MT 4 cell;
3) Testing sample: Microbicide gel prepared as Example 1, formulation 5.
   2. Testing Methods and Standards: [symbol] Technical Standard for Disinfection [symbol] (2002 Edition) Issued by The Health Department of China.
1) HIV Virus deactivation Tests, Page: 38
2) Diluted the Testing sample to 1:20; 1:40 and 1:80 solutions with distilled water.
3) Added 0.9 ml sample (1:20; 1:40; 1:80) to 0.1 ml HIV suspension solution respectively
4) The mixtures were reacted for 1 min.; 3 min.; 5 min. and 10 min. at room temperature
5) Take 0.1 ml each of the above and dilute with culture fluid to 1:1000
6) The virus concentration were titrated (96 well plate, each well contains 0.1 ml of 50000/ml MT4 cell)
7) Incubated for 7 days (37° C., 5% CO₂)
8) Added 0.1 ml of fresh culture fluid to each well at the third day
9) Observe the cell growth everyday
10) Evaluate the existence of Virus with CPE

### 3. Experimental Groups:

1) Testing group
2) Drug (sample) removed control group (1000 times diluted Virus+1000 times diluted sample)
3) Normal cell control group
4) Virus control group (Virus+culture fluid)

### 4. Results

1) HIV-1 titration was 10⁷ TCID₅₀.
2) The “drug removed control group” showed that the dilution could stop the effect of sample to the virus.
3) The testing sample at 1:20 and 1:40 dilution reacted with HIV-1 for 1 min, titrated and cultured with MT4 cell, no cell abnormality were observed.
4) The testing sample at 1:80 dilution reacted with HIV-1 for 3 min., titrated and cultured with MT4 cell, no cell abnormality were observed

### 5. Conclusion

Testing sample diluted at 1:20 (Example 1, formulation 7) and 1:40 solutions (Example 1, formulation 6) reacted with HIV-1 suspension for 1 minutes, the kill rate was 100%.

Testing sample diluted at 1:80 solution reacted with HIV-1 suspension for 3 min, the kill rate was 100%.

### EXAMPLE 11

### The Effects of the Microbicide on Treponema Pallidum

### 1. Materials

1) Treponema Pallidum: Nichols strain
2) Testing sample: Microbicide gel prepared as of Example 1, formulation 5
   2. Testing Methods and Standards [symbol] Guidelines for Pre-clinical and Clinical Research of New Drugs [symbol] (1993 Edition): “The Experimental Methods and Procedures for Anti-Treponema Pallidum”
1) Temperature: 20-25° C.
2) Sample Dilutions: 1:5; 1:10; 1:20; 1:40; 1:80; 1:100
3) Treponema Pallidum Suspension: each field≧30 Treponema Pallidum
4) Reacting time: 1 min.; 3 min.; 5 min.; 10 min.
5) Sample diluted with distilled water
6) No terminating agent was employed in the tests
7) The deactivating effects for Treponema Pallidum were recorded at different time intervals.
8) The tests repeated two times

### 3. Results

TABLE 6
The deactivating Effect of Testing Sample to Treponema Pallidum

| Treponema | React time | Sample dilution | | | | | |
|---|---|---|---|---|---|---|---|
| Pallidum | (min.) | 1:5 | 1:10 | 1:20 | 1:40 | 1:80 | 1:100 |
| Nichols strain | 1 | − | − | + | + | + | + |
| Nichols strain | 3 | − | − | − | + | + | + |
| Nichols strain | 5 | − | − | − | − | + | + |
| Nichols strain | 10 | − | − | − | − | − | + |

Note:
−: deactivated
+: not deactivated.

### 4. Conclusion

Testing sample diluted at 1:10 (Example 1, formulation 10) deactivated Treponema Pallidum within 1 min.

Testing sample diluted at 1:20 (Example 1, formulation 7) deactivated Treponema Pallidum within 3 min.

Testing sample diluted at 1:40 (Example 1, formulation 6) deactivated Treponema Pallidum within 5 min.

Testing sample diluted at 1:80 (Example 1, formulation 1 1) deactivated Treponema Pallidum within 10 min.

### EXAMPLE 12

The Effects of the Microbicide on Chlamydia trachomatis

### 1. Materials

1) Chlamydia strain: Chlamydia trachomatis E Bour type (the 10^{th} generation)
(Provided by the CDC of USA)
2) Cells: McCoy cell;
3) Culture medium: RPMI-1640 culture medium;
4) Testing sample: Microbicide gel prepared as of Example 1, formulation 5
5) Neutralizer: Diluting neutralize methods were employed, the diluents was 1640 medium with 10% bovine serum.
   2. Testing Methods and Standards: [symbol] Technical Standard for Disinfection [symbol] (2002 Edition) Issued by “The Health Department of China”

The tests followed the “Standard” with consideration of the characteristics of Chlamydia trachomatis.
1) Temperature: 25° C.
2) Concentration tested: 1:20; 1:50; 1:100; 1:200; 1:500
3) Concentration of Chlamydia trachomatis suspension: 10⁵
4) Reacted time: 1 min.; 3 min.; 5 min.; 10 min.
5) Diluting methods were used to remove the residue of sample.

### 3. Results

Killing Effects of Testing sample to Chlamydia trachomatis (Table 7)

TABLE 7
The Killing Effects of Testing sample on Chlamydia trachomatis

| | time | Dilution of Testing sample | | | | | |
|---|---|---|---|---|---|---|---|
| strain | (min.) | 1:20 | 1:50 | 1:100 | 1:200 | 1:500 | control |
| E | 1 | − | − | + | + | + | + |
| Bour | 3 | − | − | − | + | + | + |
| strain | 5 | − | − | − | + | + | + |
| | 10 | − | − | − | − | + | + |

Note:
Control: Chlamydia trachomatis growth observed;
+: there are Chlamydia trachomatis inclusion in the cell;
−: there was no Chlamydia trachomatis inclusion in the cell

### 4. Conclusion

Testing sample diluted at 1:50 (example 1, formulation 9) and reacted with Chlamydia trachomatis suspension for 1 minute killed Chlamydia trachomatis.

### EXAMPLE 13

### The Effects of the Microbicide on Ureaplasma Urealyticum

### 1. Materials

Testing strain: Ureaplasma Urealyticum: international standard strain; used 6^{th} generation

Culture medium: High efficiency Ureaplasma culture medium;

Testing sample: Microbicide gel prepared as of Example 1, formulation 5.

2. Testing Methods and Standards: [symbol] Technical Standard for Disinfection [symbol] (2002 Edition) Issued by “The Health Department of China”

The tests followed the “Standard” with consideration of the characteristics of Ureaplasma Urealyticum
1) Testing Temperature: 25° C.˜28° C.
2) Concentration of sample dilution: 1:100; 1:200; 1:500; 1:1000
3) Concentration of Ureaplasma Urealyticum: 10⁷
4) Treatment: Ureaplasma Urealyticum suspension:diluted sample=1:9
5) Reacted time: 1 min.; 3 min.; 5 min.; 10 min.
6) Diluting methods were used to remove the residue of drug
7) Tests repeated for two times

### 3. Results

The Killing Effect of Microbicide gel to Ureaplasma Urealyticum (Table 8)

TABLE 8
The Effects of Testing sample on Ureaplasma Urealyticum

| | |
|---|---|
| time | Dilutions of Testing Sample |

| (min.) | 1:100 | 1:200 | 1:500 | 1:1000 | control |
|---|---|---|---|---|---|
| 1 | − | − | + | + | + |
| 3 | − | − | + | + | + |
| 5 | − | − | + | + | + |
| 10 | − | − | + | + | + |

Note:
Control: Ureaplasma Urealyticum growth observed;
+: Ureaplasma Urealyticum growth observed;
−: No Ureaplasma Urealyticum growth observed.

### 4. Conclusion

The testing results showed: Testing sample diluted for 200 times (Example 1, formulation 4) and reacted with Ureaplasma Urealyticum suspension for I minute, killed Ureaplasma Urealyticum.

### EXAMPLE 14

The Effects of the Microbicide on Neisseria gonorrhoeae

### 1. Materials

1) Testing strain: Standard strain G, provided by WHO
2) Culture medium: 10% blood dish;
3) Testing sample: Microbicide gel prepared as of Example 1, formulation 5.
   2. Testing Methods and Standards: [symbol] Technical Standard for Disinfection [symbol] (2002 Edition) Issued by “The Health Department of China”

The tests followed the “Standard” with consideration of the characteristics of Neisseria gonorrhoeae
1) Temperature: 25° C.˜28° C.
2) Concentration of sample prepared: 1:25; 1:50; 1:100; 1:250; 1:500
3) Neisseria gonorrhoeae suspension: 10⁷
4) Treatment: Neisseria gonorrhoeae suspension:diluted sample solution=1:1
5) Treatment time: 1 min.; 3 min.; 5 min.; 10 min.
6) Residue drug were removed with diluting methods
7) Tests repeated for three times

### 3. Results

The Killing Effect of Microbicide gel to Ureaplasma Urealyticum (Table 9)

TABLE 9
The Killing Effects of Testing Sample to Neisseria gonorrhoeae

| | |
|---|---|
| React time | Dilutions of Testing sample |

| (min.) | 1:50 | 1:100 | 1:200 | 1:500 | control |
|---|---|---|---|---|---|
| 1 | − | − | + | + | + |
| 3 | − | − | + | + | + |
| 5 | − | − | + | + | + |
| 10 | − | − | − | + | + |

Note:
+: growth of Neisseria gonorrhoeae observed
−: No growth of Neisseria gonorrhoeae

### 4. Conclusion

The testing results showed: Testing sample diluted for 100 times (example 1, formulation 8) and reacted with Neisseria gonorrhoeae suspension for 1 minute killed Neisseria gonorrhoeae.

### EXAMPLE 15

The Effects of the Microbicide on Trichomonas vaginalis

### 1. Materials

1) Testing strain: Clinical isolated Trichomonas vaginalis strain
2) Culture medium: liver infusion medium;
3) Testing sample: Microbicide gel prepared as of Example 1, formulation 5.
   2. Testing Methods and Standards: [symbol] Technical Standard for Disinfection [symbol] (2002 Edition) Issued by “The Health Department of China”

The tests followed the “Standard” with consideration of the characteristics of Trichomonas vaginalis
1) Trichomonas vaginalis suspension: 10⁵/ml
2) Concentration of sample prepared: 1:50; 1:100; 1:200
3) Treatment: Trichomonas vaginalis suspension:diluted sample solution=1:1
4) Reaction time: 1 min.; 3 min.; 5 min.; 10 min.
5) Temperature: 20˜25° C.
6) Residue drug were removed with diluting methods
7) Tests repeated for two times

### 3. Results

TABLE 10
The Killing Effects of Testing Sampleto Trichomonas vaginalis

| React time | Dilutions of Testing sample | | | |
|---|---|---|---|---|
| (min.) | 1:100 | 1:200 | 1:400 | control |
| 1 | − | ± | + | + |
| 3 | − | ± | + | + |
| 5 | − | ± | + | + |
| 10 | − | − | + | + |

Note:
+: Active Trichomonas vaginalis
±: Most Trichomonas vaginalis were killed, a few active left
−: Trichomonas vaginalis were not active and were dissolved

### 4. Conclusion

The testing results showed: Testing sample diluted for 100 times (example 1, formulation 8) and reacted with Trichomonas vaginalis suspension for 1 minute, killed Trichomonas vaginalis.

### EXAMPLE 16

### The Effects of the Microbicide on Herpes Simplex Virus

### 1. Materials

1) Testing strain: Herpes Simplex type II strain. Standard international strain, provided by the Drug and bio-product office of the Health Department of China.
2) Cell: Vero cell strain, introduced by the Cell biology Institute of Shanghai
3) Culture medium: 1640 culture medium
4) Testing sample: Microbicide gel prepared as of Example 1, formulation 5.
   2. Testing Methods and Standards: [symbol] Technical Standard for Disinfection [symbol] (2002 Edition) Issued by “The Health Department of China”

The tests followed the “Standard” with consideration of the characteristics of Herpes Simplex
1) Virus suspension: 5×10⁴/ml
2) Concentration of sample prepared: 1:20; 1:40; 1:50
3) Reaction time: 1 min.; 3 min.; 5 min.; 10 min.
4) Temperature: 20-25° C.
5) Residue drug were removed with diluting methods (2% bovine serum culture medium as diluents)
6) Tests repeated for two times

### 3. Results

The Killing Effect of Microbicide gel to Herpes Simplex Virus (Table 11)

TABLE 11
The Effects of Testing Example on Herpes Simplex Virus

| React time | Dilutions of Testing sample | | | |
|---|---|---|---|---|
| (min.) | 1:20 | 1:40 | 1:50 | Control |
| 1 | − | ± | + | + |
| 3 | − | ± | + | + |
| 5 | − | ± | + | + |
| 10 | − | ± | + | + |

Note:
+: Virus growth observed, infected cell > 50%
±: Virus growth observed, infected cell < 10%
−: No virus growth

### 4. Conclusion

The testing results showed: Testing sample diluted for 20 times (Example 1, formulation 7) and reacted with Herpes Simplex Virus. Herpes Simplex Virus was killed with 20 times dilution of the sample in 1 minute.

### EXAMPLE 17

The Effects of the Microbicide on Staphylococcus aureus

### 1. Materials

1) Testing strain: Staphylococcus aureus ATCC6538, 5-6^{th} generations, provided by the reserve center of China microbial reservation committee.
2) Testing medium carrier: Sterilized filter paper
3) Testing sample: Microbicide gel prepared as of Example 1, formulation 5.
   2. Testing Methods and Standards: [symbol] Technical Standard for Disinfection [symbol] (2002 Edition)

GB 15979-2002 [symbol] Technical Standard for Testing Disinfection Product [symbol] “Procedures for testing bactericide effect”

### 3. Results

The bactericide effect of testing sample to Staphylococcus aureus

At 20±1° C. temperature, three repeated tests showed: The testing sample reacted with Staphylococcus aureus for 1 minute the average bactericide rate was 95.70%. (table 12)

TABLE 12
The Bactericide Effect of Testing Sample to Staphylococcus aureus

| | | | | Average | |
|---|---|---|---|---|---|
| Time | Bactericide rates of three tests | | | bactericide | Control |
| (min.) | First test | Second test | Third test | rate (%) | (CFU/pc.) |
| 1 | 92.13 | 96.87 | 98.10 | 95.70 | 1.28 × 10⁴ |
| 3 | 99.64 | 100.00 | 99.58 | 99.74 | 1.25 × 10⁴ |
| 5 | 99.72 | 100.00 | 99.95 | 99.89 | 1.23 × 10⁴ |

### 4. Conclusion

Testing sample (Example 1, formulation 5) reacted with Staphylococcus aureus for 1 minute the average bactericide rate was 95.70%.

### EXAMPLE 18

The Effects of the Microbicide on Candida albicans

### 1. Materials

1) Testing strain: Candida albicans ATCC 10231, the 5-6 generations; Provide by the “Reserve Center of Chinese Microbial Reservation Committee”.
2) Testing sample: Microbicide gel prepared as of Example 1, formulation 5.
   2. Testing Methods and Standards: [symbol] Technical Standard for Disinfection [symbol] (2002 Edition) “Procedures for testing bactericide effect” GB15979-2002 [symbol] Technical Standard for Testing Disinfection Product

[symbol]

Temperature: 20±1° C.

### 3. Results

The bactericide effect of Testing sample to Candida albicans ATCC10231 At 20±1° C. temperature, three repeated tests showed: Testing material reacted with Candida albicans ATCC10231 for 1 minutes, the average bactericide rate was 95.47%. (Table 13)

TABLE 13
The bactericide effect of Testing Sample to Candida albicans

| | | | | Average | |
|---|---|---|---|---|---|
| Time | Bactericide rates of three tests | | | bactericide | Control |
| (min.) | First test | Second test | Third test | rate (%) | (CFU/pc.) |
| 1 | 97.95 | 93.81 | 94.65 | 95.47 | 3.07 × 10⁴ |
| 3 | 99.66 | 99.96 | 99.30 | 99.53 | 2.99 × 10⁴ |
| 5 | 99.91 | 99.88 | 99.75 | 99.84 | 2.96 × 10⁴ |

### 4. Conclusion

Testing sample reacted with Candida albicans for 1 minutes, the average bactericide rate was 95.47%. The Microbicidal Gel has bactericide effect to Candida albicans.

### EXAMPLE 19

The Effects of the Microbicide on Eschetichia coli

### 1. Materials

1) Testing strain: Eschetichia coli 8099, the 5-6 generations; provided by the “Reserve Center of Chinese Microbial Reservation Committee”.
2) Testing sample: Microbicide gel prepared as of Example 1, formulation 5.
   2. Testing Methods and Standards: [symbol] Technical Standard for Disinfection [symbol] (2002 Edition) “Procedures for testing bactericide effect” GB15979-2002 [symbol] Technical Standard for Testing Disinfection Product

[symbol]

### 3. Results

The bactericide effect of Testing sample to Eschetichia coli:

At 20±1° C. temperature, three repeated tests showed: the sample gel reacted with Eschetichia coli for 1 minutes, the average bactericide rate was 99.97%. (Table. 14)

TABLE 14
The Bactericide Effect of Testing Sample to Eschetichia coli

| | | | | Average | |
|---|---|---|---|---|---|
| Time | Bactericide rates of three tests | | | bactericide | Control |
| (min.) | First test | Second test | Third test | rate (%) | (CFU/pc.) |
| 1 | 99.96 | 100.00 | 99.95 | 99.97 | 1.30 × 10⁴ |
| 3 | 100.00 | 100.00 | 100.00 | 100.00 | 1.22 × 10⁴ |
| 5 | 100.00 | 100.00 | 100.00 | 100.00 | 1.20 × 10⁴ |

### 4. Conclusion

Testing sample reacted with Eschetichia coli for 1 minutes, the average bactericide rate was 99.97%.

### EXAMPLE 20

Spermicidal Effects of the Microbicide Solution (in vitro).

### 1. Materials

1) Human sperm: Normal sperms obtained from three health Adults Testing
2) Sample: Microbicide solution prepared as of Example 1, formulation 37.

### 2. Methods

1) Testing sample was tested undiluted (1:1), diluted 5 times (1:5) and 10 times (1:10). Mix the prepared samples with equal volume of fresh sperm (0.5 ml sample and 0.5 ml sperm)
2) Determine the minimum concentration and time of demobilize all the sperms
3) Use saline as negative control.
4) Repeat the tests for three times.

### 3. Results

The undiluted solution reacted with sperms for 30 seconds. The survival rate of sperms was zero. Table 15, 16 and 17:

TABLE 15
Testing Results on Sperm Sample 1:

| | | sample | | |
|---|---|---|---|---|
| | | concentration | | |
| Time | | 1:1 | 1:5 | 1:10 |
| 30 sec | Sperm count (million/ml) | 23 | 23 | 23 |
| | Active rate (%) | 0 | 50 | 60 |
| 1 min | Sperm count (million/ml) | 23 | 22 | 22 |
| | Active rate (%) | 0 | 45 | 45 |
| 2 min | Sperm count (million/ml) | 22 | 22 | 22 |
| | Active rate (%) | 0 | 42 | 45 |
| 3 min | Sperm count (million/ml) | 23 | 23 | 23 |
| | Active rate (%) | 0 | 40 | 40 |

| | Before test | Control/saline |
|---|---|---|
| Sperm count (million/ml) | 45 | 23 |
| Active rate (%) | 82 | 72 |

TABLE 16
Testing Results on Sperm Sample 2:

| | | sample | | |
|---|---|---|---|---|
| | | concentration | | |
| Time | | 1:1 | 1:5 | 1:10 |
| 30 sec | Sperm count (million/ml) | 13.5 | 13.5 | 13.5 |
| | Active rate (%) | 0 | 50 | 50 |
| 1 min | Sperm count (million/ml) | 13.5 | 13.4 | 13.4 |
| | Active rate (%) | 0 | 50 | 50 |
| 2 min | Sperm count (million/ml) | 13.3 | 13.4 | 13.5 |
| | Active rate (%) | 0 | 45 | 50 |
| 3 min | Sperm count (million/ml) | 13.2 | 13.2 | 13.3 |
| | Active rate (%) | 0 | 40 | 50 |

| | Before test | Control/saline |
|---|---|---|
| Sperm count (million/ml) | 25 | 13.5 |
| Active rate (%) | 62 | 62 |

TABLE 17
Testing Results on Sperm Sample 3

| | | sample | | |
|---|---|---|---|---|
| | | concentration | | |
| Time | | 1:1 | 1:5 | 1:10 |
| 30 sec | Sperm count (million/ml) | 29.7 | 29.5 | 29.4 |
| | Active rate (%) | 0 | 60 | 61 |
| 1 min | Sperm count (million/ml) | 29.6 | 29.0 | 29.0 |
| | Active rate (%) | 0 | 58 | 59 |
| 2 min | Sperm count (million/ml) | 29.0 | 29.0 | 29.0 |
| | Active rate (%) | 0 | 58 | 58 |
| 3 min | Sperm count (million/ml) | 28.5 | 28.5 | 28.5 |
| | Active rate (%) | 0 | 58 | 58 |

| | Before test | Control/saline |
|---|---|---|
| Sperm count (million/ml) | 58 | 30 |
| Active rate (%) | 75 | 73 |

### 4. Test Results

The undiluted solution reacted with equal volume sperm for 30 sec, 1 min, 2 min, 3 min. The sperm survive rate was zero. The spermicidal rate of testing microbicide solution was 100% in 30 seconds (in vitro). Saline reacted with equal volume sperm, the sperm count and active rate was not influenced.

### 5. Conclusion

The spermicidal rate of testing sample was 100% in 30 seconds (in vitro).

### EXAMPLE 21

Spermicidal Effects of the Microbicide Gel (in vitro)

### 1. Materials

1) Human sperm: Normal sperms obtained from three health Adults
2) Testing sample: Microbicide gel prepared as of Example 1, formulation 5.

### 2. Methods

1) Dilute the Testing sample to different dilute concentrations (1:10, 1:5, 1:1)
2) Mix the diluted samples with equal volume fresh sperm (0.5 ml sample and 0.5 ml sperm)
3) Determine the minimum concentration and time of demobilize all the sperms
4) Use saline as negative control
5) Repeat the tests for three times

### 3. Results

The undiluted gel reacted with sperms for 30 seconds, the survive rate of sperms was zero;

The gel diluted at 1:1, 1:5 and reacted with equal volume sperm for 30 sec., 1 min., 2 min. and 3 min. The sperm survive rate was zero;

The gel diluted at 1:10 and reacted with equal volume sperm for 30 sec. The sperm survive rate was 10%;

The 1:10 gel dilution reacted with sperm for 1 min., 2 min. and 3 min. respectively the sperm survive rate was zero.

The 1:10 dilution deactivated all the sperms for 60 seconds. The survive test showed the sperm survive rate was zero.

TABLE 18
Testing Results on Sperm Sample 1:

| | | | sample dilution | | | |
|---|---|---|---|---|---|---|
| Time | | | gel | 1:1 | 1:5 | 1:10 |
| 30 | sec | Sperm count (million/ml) | 12.3 | 11.4 | 11.0 | 10.1 |
| | | Active rate (%) | 0 | 0 | 0 | 10 |
| 1 | min | Sperm count (million/ml) | — | 10.0 | 9.8 | 9.7 |
| | | Active rate (%) | — | 0 | 0 | 0 |
| 2 | min | Sperm count (million/ml) | — | 9.6 | 9.8 | 9.5 |
| | | Active rate (%) | — | 0 | 0 | 0 |
| 3 | min | Sperm count (million/ml) | — | 9.5 | 9.4 | 9.5 |
| | | Active rate (%) | — | 0 | 0 | 0 |

| | Before test | Control/saline |
|---|---|---|
| Sperm count (million/ml) | 20.1 | 12.5 |
| Active rate (%) | 65 | 64.5 |

TABLE 19
Testing Results on Sperm Sample 2:

| | | | Sample concentration | | | |
|---|---|---|---|---|---|---|
| Time | | | gel | 1:1 | 1:5 | 1:10 |
| 30 | sec | Sperm count (million/ml) | 20.0 | 19.2 | 19.0 | 18.9 |
| | | Active rate (%) | 0 | 0 | 0 | 5 |
| 1 | min | Sperm count (million/ml) | — | 18.4 | 19.0 | 18.7 |
| | | Active rate (%) | — | 0 | 0 | 0 |
| 2 | min | Sperm count (million/ml) | — | 18.6 | 18.6 | 18.5 |
| | | Active rate (%) | — | 0 | 0 | 0 |
| 3 | min | Sperm count (million/ml) | — | 18.0 | 18.0 | 17.9 |
| | | Active rate (%) | — | 0 | 0 | 0 |

| | Before test | Control/saline |
|---|---|---|
| Sperm count (million/ml) | 36.3 | 20.9 |
| Active rate (%) | 70 | 69 |

TABLE 20
Testing Results on Sperm Sample 3:

| | | | Sample concentration | | | |
|---|---|---|---|---|---|---|
| Time | | | gel | 1:1 | 1:5 | 1:10 |
| 30 | sec | Sperm count (million/ml) | 14.3 | 14.2 | 14.2 | 14.0 |
| | | Active rate (%) | 0 | 0 | 0 | 15 |
| 1 | min | Sperm count (million/ml) | — | 13.8 | 13.7 | 14.0 |
| | | Active rate (%) | — | 0 | 0 | 0 |
| 2 | min | Sperm count (million/ml) | — | 13.6 | 13.5 | 13.3 |
| | | Active rate (%) | — | 0 | 0 | 0 |
| 3 | min | Sperm count (million/ml) | — | 13.2 | 13.1 | 13.1 |
| | | Active rate (%) | — | 0 | 0 | 0 |

| | Before test | Control/saline |
|---|---|---|
| Sperm count (million/ml) | 23.2 | 15.1 |
| Active rate (%) | 75 | 75 |

### 4. Conclusion

The spermicidal rate of testing microbicide gel was 100% in 30 seconds (in vitro), the minimum effective dilution was 1:5.

The spermicidal rate of testing microbicidal gel was 100% in 60 seconds (in vitro), the minimum effective dilution was 1:10.

What is claimed is:

1. A broad spectrum and highly efficient microbicidal composition comprising essentially: 0.003 to 3.0% (by weight) of N-cocoyl amino acid pyrrolidone salts and from 10 to 99.997% (by weight) of water.

2. The composition according to claim 1, wherein said composition can be added a plant polysaccharide.

3. A composition according to claim 1, wherein the amino acid group of the N-cocoyl amino acid pyrrolidone salts, can be selected from a group of an arginine, lysine, and histidine; homolysine, or unnatural amino acid residue bearing a positive charge, and di or tri-peptide bearing a positive charge group.

4. The composition according to claim 1, wherein said water can be de-ionized water, distilled water, or pure water; and wherein said water can also be substituted by or mixed with glycerin, glycerol-gelatin, ethanol, propylene glycol, polyethylene glycol or other compatible solvents, emulsions, excipients, and stabilizer.

5. The composition according to claim 1 or claim 2, wherein said plant polysaccharides can be freeze-dried powder of Aloe Vera gel, or other forms of Aloe Vera polysaccharides.

6. The composition according to claim 1 or claim 2, wherein the compositions can be prepared in a form selected from the group consisting of a suppository, gel, cream, salve, jelly, spray, liquid, paste, aerosol, lotion, tablet, foam, film, and mist.

7. The composition according to claim 1 or claim 2, wherein the compositions can typically be applied to the external genital organs, vagina, anorectic region and rectum using an appropriate dosage form selected from the group consisting of a suppository, gel, cream, salve, jelly, spray, liquid, paste, aerosol, lotion, tablet, foam, film, and mist with an appropriate an applicator.

8. The composition according to claim 1 or claim 2, wherein the composition can be used for inhibiting the activity of sexually transmitted diseases pathogens, bacteria, viruses, fungi, and protozoan.

9. The composition according to claim 1 or claim 2, wherein the composition can be used for deactivating human sperms.

10. The composition according to claim 1 or claim 2, wherein the composition can be used in vaginal mucous membrane for preventing and treating sexually transmitted diseases (STDs) through inhibiting the activity of the STDs pathogens.

11. The composition according to claim 1 or claim 2, wherein the composition can be used in vaginal mucous membrane for preventing and treating women's diseases caused by birth tract infections through inhibiting the activity of bacteria, viruses, fungi, and protozoan.

12. The composition according to claim 1 or claim 2, wherein the composition can be used in vagina, exterior sex organs and or rectum for preventing Aids through inhabiting the activity of HIV.

13. The composition according to claim 1 or claim 2, wherein the composition can be used on skin and mucous to treat skin and mucous diseases.

14. The composition according to claim 1 or claim 2, wherein the composition can be used as disinfectant for the disinfection of skin and mucous surfaces.

15. The composition for the microbicide according to claim 1 or claim 2, wherein the composition can be used for reducing the transmission of STDs pathogens to health care providers, laboratory personnel, and related persons who may touch biological samples and specimens.

16. The composition according to claim 1 or claim 2, wherein the composition can be used to animals as disinfectant for the disinfection of skin and mucous surfaces.

17. The composition according to claim 1 or claim 2, wherein the composition can be used in vagina to prevent unwanted pregnancy.
